(19) United States
(12) Patent Application Publication
(10) Pub. No.: US 2006/0270859 A1
(43) Pub. Date: Nov. 30, 2006

(54) Duloxetine HCl polymorphs

(76) Inventors: Santiago Ini, Haifa (IL); Yaron Shmuel, Hedera (IL); Tamas Koltai, Natania (IL); Amir Gold, Herzelia (IL)

Correspondence Address: KENYON & KENYON LLP, ONE BROADWAY, NEW YORK, NY, 10004, US

(21) Appl. No.: 11/341,923

(22) Filed: Jan. 27, 2006

Related U.S. Application Data
(60) Provisional application No. 60/647,888, filed on Jan. 27, 2005.

(51) Int. Cl. C07D 333/22

(52) U.S. Cl. 549/76

(57) ABSTRACT

A crystalline form of duloxetine hydrochloride, pharmaceutical compositions of the crystalline form of duloxetine hydrochloride, and methods of preparing the crystalline form of duloxetine hydrochloride are provided.

### RELATED APPLICATIONS

This application claims benefit of U.S. Provisional Patent Applications No. 60/647,888, filed Jan. 27, 2005, the contents of which are incorporated herein in their entirety.

### FIELD OF INVENTION

The present invention is directed to solid states of duloxetine HCI and methods of preparation thereof.

### BACKGROUND OF THE INVENTION

Duloxetine is a dual reuptake inhibitor of the neurotransmitters serotonin and norepinephrine, and has been found to have application for the treatment of stress urinary incontinence (SUI), depression and pain management.

A method for the synthesis of duloxetine HCI is disclosed in U.S. Pat. No. 5,362,886. However, that patent does not disclose any particular crystalline form of duloxetine HCl. When duloxetine HCl is prepared according to Preparation 2 of U.S. Pat. No. 5,362,886, an anhydrous crystalline form is obtained. As used herein, the term “Form A” refers to the anhydrous crystalline form of duloxetine HCl obtained using Preparation 2 of U.S. Pat. No. 5,362,886. Duloxetine HCl is available commercially as Cymbaltao®, which contains Form A as the active ingredient.

An amorphous form of duloxetine HCl is disclosed in WO 2005/019199.

Polymorphism, the occurrence of different crystal forms, is a property of some molecules and molecular complexes. A single molecule, like duloxetine HCl, may give rise to a variety of crystalline forms having distinct crystal structures and physical properties like melting point, x-ray diffraction pattern, infrared absorption fingerprint, and solid state NMR spectrum. One crystalline form may give rise to thermal behavior different from that of another crystalline form. Thermal behavior can be measured in the laboratory by such techniques as capillary melting point, thermogravimetric analysis (“TGA”), and differential scanning calorimetry (“DSC”), which have been used to distinguish polymorphic forms.

The difference in the physical properties of different crystalline forms results from the orientation and intermolecular interactions of adjacent molecules or complexes in the bulk solid. Accordingly, polymorphs are distinct solids sharing the same molecular formula yet having distinct advantageous physical properties compared to other crystalline forms of the same compound or complex.

One of the most important physical properties of pharmaceutical compounds is their solubility in aqueous solution, particularly their solubility in the gastric juices of a patient. For example, where absorption through the gastrointestinal tract is slow, it is often desirable for a drug that is unstable to conditions in the patient's stomach or intestine to dissolve slowly so that it does not accumulate in a deleterious environment. Different crystalline forms or polymorphs of the same pharmaceutical compounds can and reportedly do have different aqueous solubilities.

The discovery of new polymorphic forms of a pharmaceutically useful compound provides a new opportunity to improve the performance characteristics of a pharmaceutical product. It enlarges the repertoire of materials that a formulation scientist has available for designing, for example, a pharmaceutical dosage form of a drug with a targeted release profile or other desired characteristic. There is a need in the art for polymorphic forms of duloxetine HCl.

### SUMMARY OF THE INVENTION

In one embodiment, the present invention provides crystalline duloxetine HCl, herein defined as Form B, characterized by X-ray powder diffraction peaks at about 11.1, 12.1, 14.9, 21.6 and 24.2 degrees two-theta ±0.2 degrees two-theta.

In another embodiment, the present invention provides a method of preparing duloxetine HCl crystal Form B, comprising providing a solution of duloxetine HCl in water and a solvent selected from the group consisting of C₁₋₄ alcohols, and removing the solvent to obtain duloxetine HCl crystal Form B.

In another embodiment, the present invention provides a process of preparing purely amorphous form of duloxetine HCl, comprising spray drying a solution of duloxetine HCl in a solvent selected from the group consisting of C₁₋₄ alcohols, where the inlet temperature is ambient, and the outlet temperature is less than the inlet temperature.

In another embodiment, the present invention provides pharmaceutical compositions comprising duloxetine HCl crystal Form B.

### BRIEF DESCRIPTION OF THE FIGURES

FIG. 1 illustrates the powder X-ray diffraction pattern for duloxetine HCl Form A.

FIG. 2 illustrates the powder X-ray diffraction pattern for duloxetine HCl Form B.

FIG. 3 illustrates the IR spectrum for duloxetine HCl Form A from 4000 to 400 cm⁻¹.

FIG. 4 illustrates the IR spectrum for duloxetine HCl Form A from 4000 to 2000 cm⁻¹.

FIG. 5 illustrates the IR spectrum for duloxetine HCl Form A from 2000 to 1000 cm⁻¹.

FIG. 6 illustrates the IR spectrum for duloxetine HCl Form A from 1000 to 400 cm⁻¹.

FIG. 7 illustrates the IR spectrum for duloxetine HCl Form B from 4000 to 400 cm⁻¹.

FIG. 8 illustrates the IR spectrum for duloxetine HCl Form B from 4000 to 2000 cm⁻¹.

FIG. 9 illustrates the IR spectrum for duloxetine HCl Form B from 2000 to 1000 cm⁻¹.

FIG. 10 illustrates the IR spectrum for duloxetine HCl Form B from 1000 to 400 cm⁻¹.

FIG. 11 illustrates the Raman spectrum for duloxetine HCl Form A from about 3500 to about 50 cm⁻¹.

FIG. 12 illustrates the Raman spectrum for duloxetine HCl Form A from about 3500 to 1500 cm⁻¹.

FIG. 13 illustrates the Raman spectrum for duloxetine HCl Form A from 1500 to 750 cm⁻¹.

FIG. 14 illustrates the Raman spectrum for duloxetine HCl Form A from 750 to 50 cm⁻¹.

FIG. 15 illustrates the Raman spectrum for duloxetine HCl Form B from about 3500 to about 50 cm⁻¹.

FIG. 16 illustrates the Raman spectrum for duloxetine HCl Form B from about 3500 to 1500 cm⁻¹.

FIG. 17 illustrates the Raman spectrum for duloxetine HCl Form B from 1500 to 750 cm⁻¹.

FIG. 18 illustrates the Raman spectrum for duloxetine HCl Form B from 750 to 50 cm⁻¹.

FIG. 19 illustrates the powder X-ray diffraction pattern for the purely amorphous form of duloxetine HCl.

### DETAILED DESCRIPTION OF THE INVENTION

As used herein the term “anhydrous” refers to duloxetine HCl containing not more than 1% water/solvent by weight.

As used herein, the term “purely amorphous” in reference to duloxetine hydrochloride, refers to non-crystalline duloxetine HCl. Preferably, the purely amorphous duloxetine hydrochloride contains less than about 5 percent crystalline forms, more preferably, less than about 3 percent, and, most preferably, less than about 1 percent, as measured as area percentage of peaks present in the XRD diffractogram.

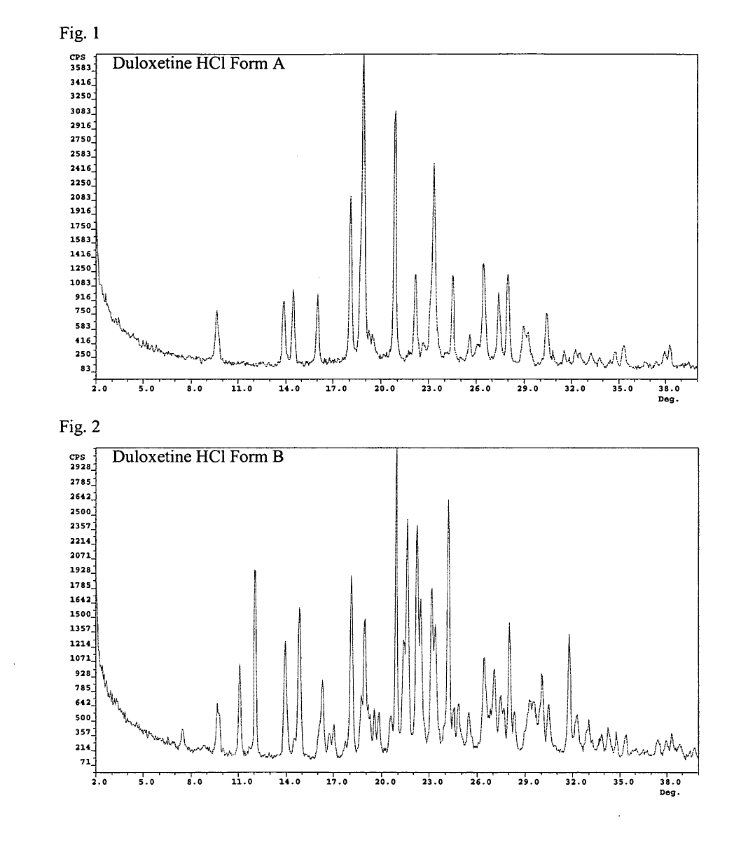

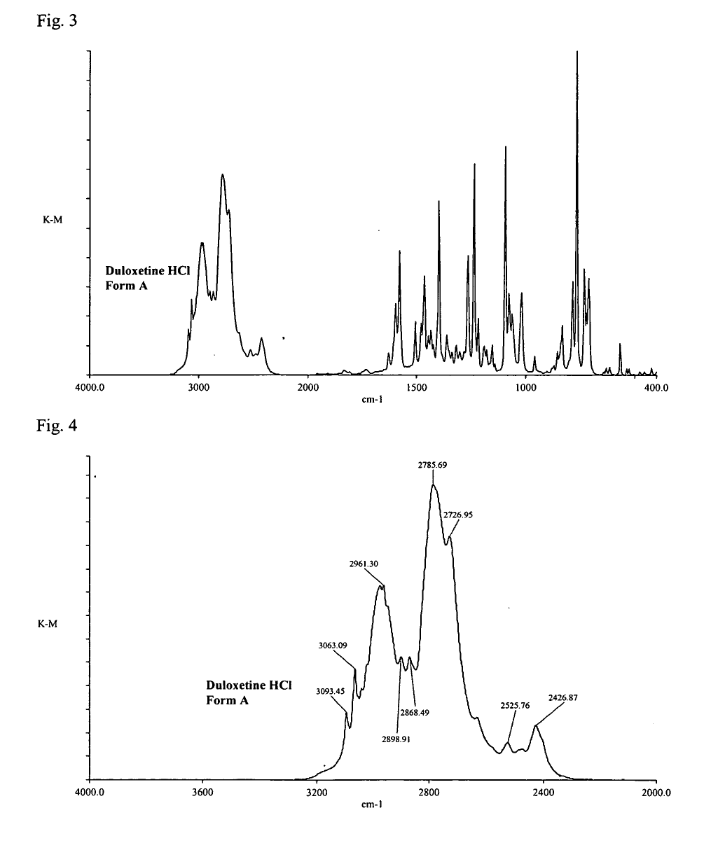

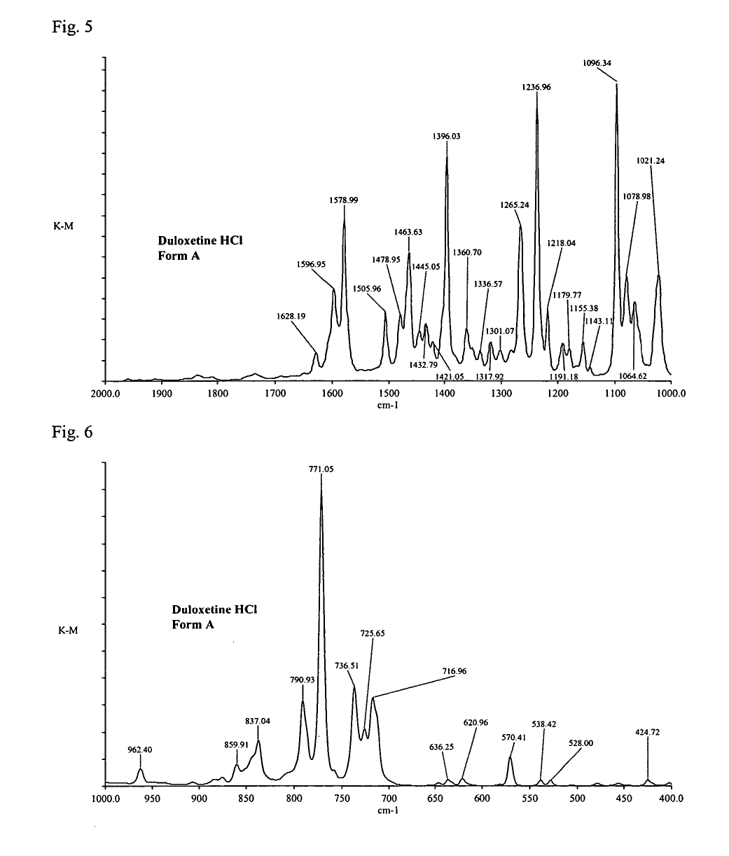

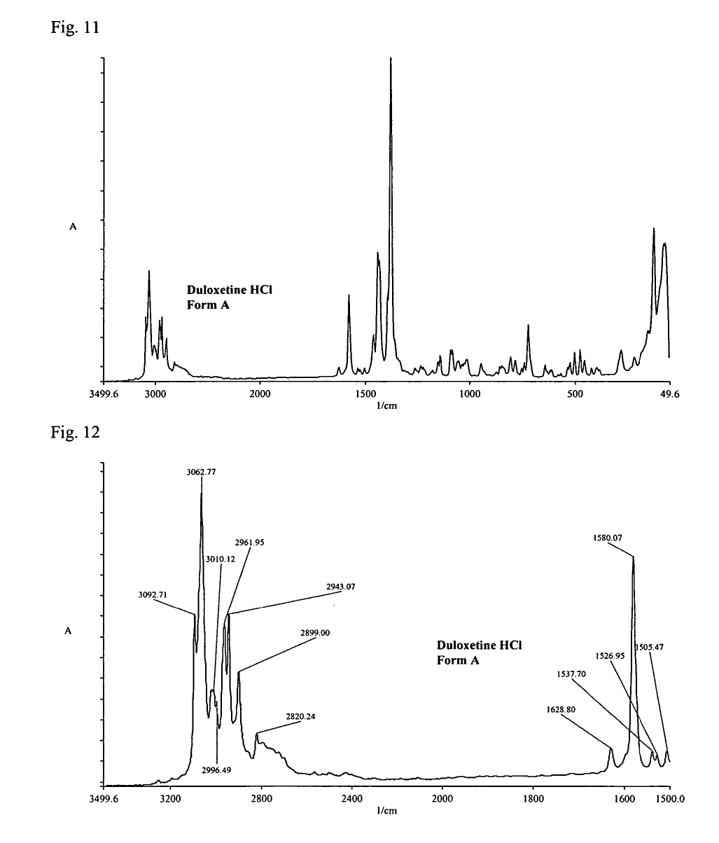

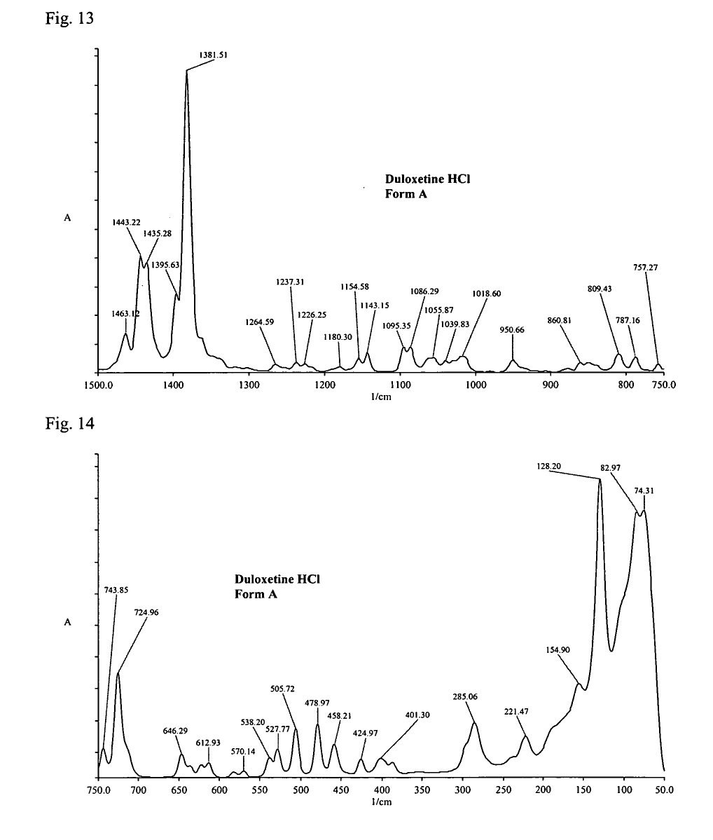

As discussed above, preparation of duloxetine HCl using prior art processes provides an anhydrous crystalline form of duloxetine HCl in a form referred to herein as Form A. Crystals of Form A duloxetine HCl were analyzed using an X-ray diffraction (XRD) diffractometer, a Fourier Transform Infrared (FTIR) spectrometer, and a Fourier Transform Raman (FTRaman) spectrometer. The XRD pattern of Form A obtained in the XRD analysis is illustrated in FIG. 1, the FTIR spectrum of Form A is illustrated in FIGS. 3 to 6, and the FTRaman spectrum of Form A is illustrated in FIGS. 11 to 14.

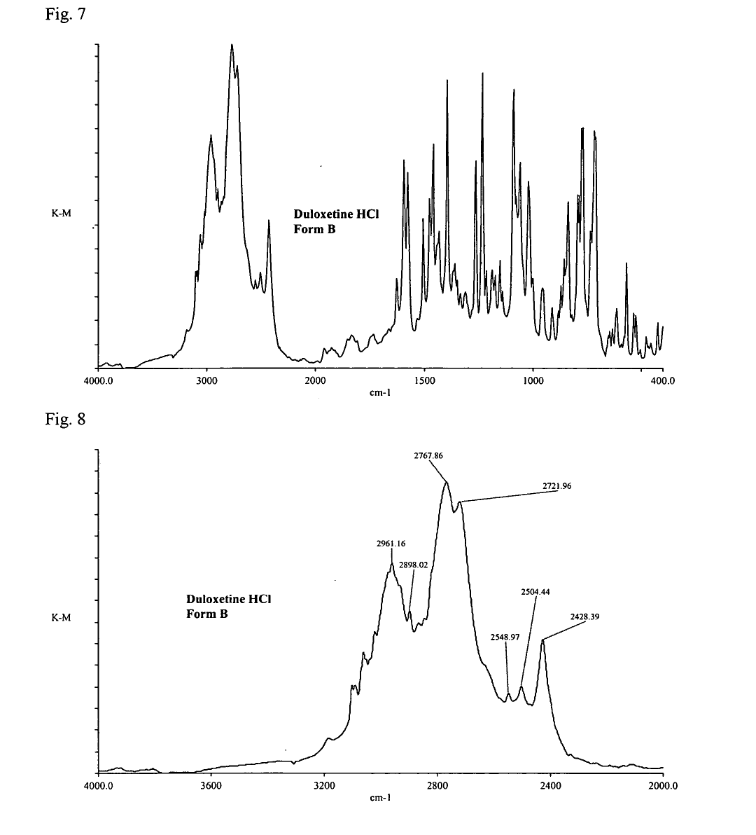

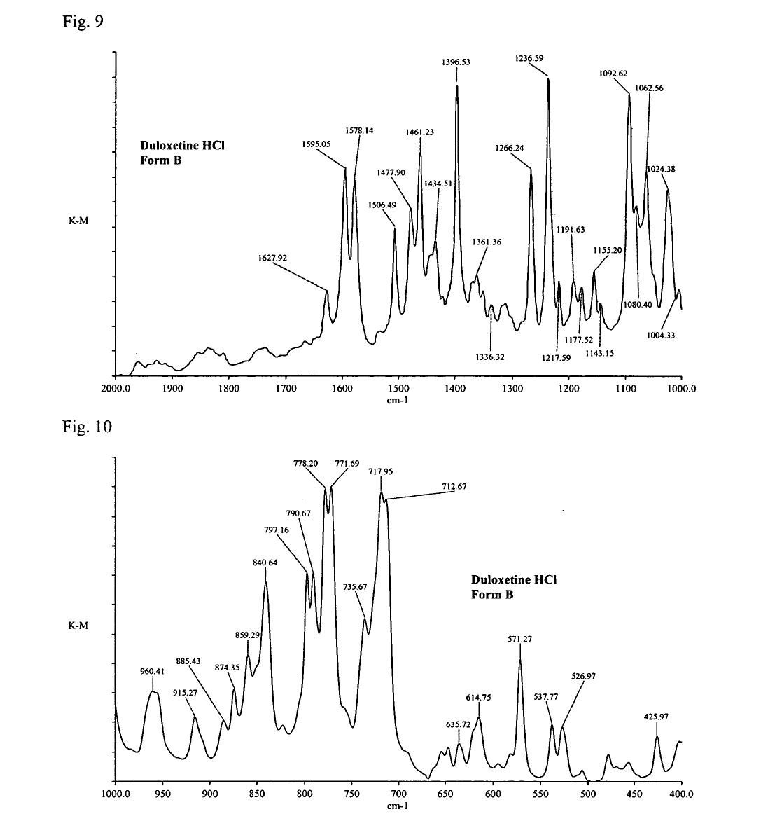

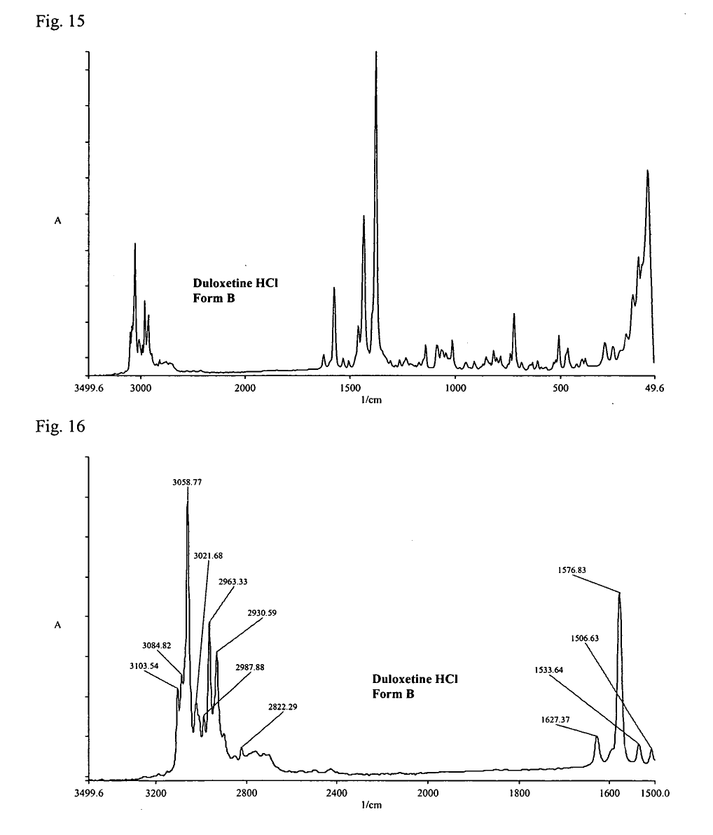

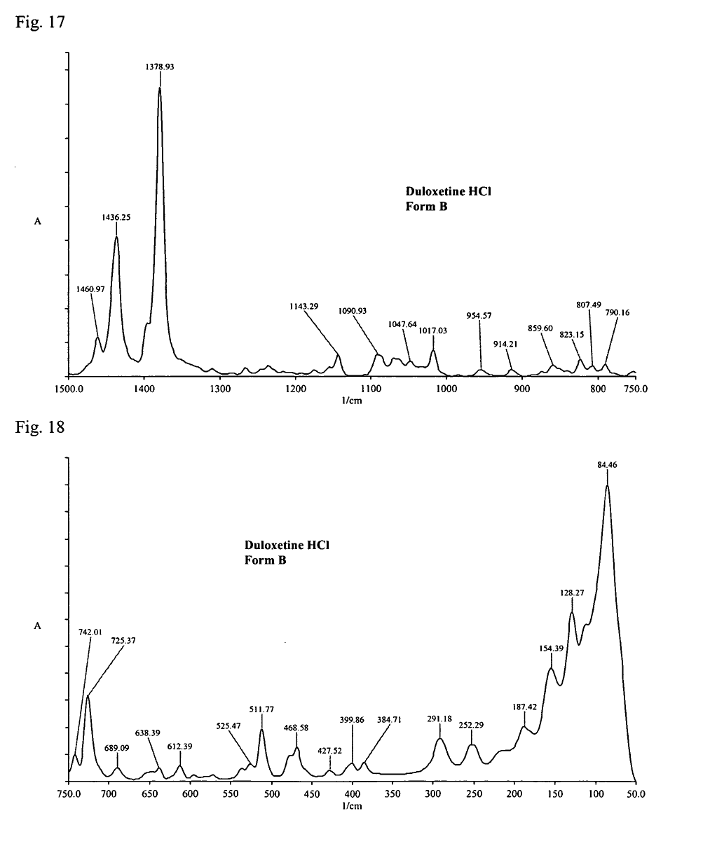

In one embodiment, the present invention provides crystalline duloxetine HCl, herein defined as Form B. Form B of duloxetine HCl is characteristically different from Form A, as demonstrated by its XRD pattern, illustrated in FIG. 2, its FTIR spectrum, illustrated in FIGS. 7 to 10, and its FTRaman spectrum, illustrated in FIGS. 15 to 18.

Duloxetine HCl crystal Form B in accordance with the invention is characterized by X-ray powder diffraction peaks at about 11.1, 12.1, 14.9, 21.6 and 24.2 degrees two-theta ±0.2 degrees two-theta. The crystalline form may be further characterized by a X-ray powder diffraction pattern with peaks at about 16.3 and 27.1 ±0.2 degrees two-theta

Duloxetine HCl crystal Form B in accordance with the invention can also be characterized by a weight loss measured by thermal gravimetric analysis (TGA) of about 0.3 percent by weight.

Duloxetine HCl crystal Form B in accordance with the invention can also be characterized by an FTIR spectrum with characteristic peaks at about 1093, 1024, 797, and 778 cm⁻¹.

Duloxetine HCl crystal Form B in accordance with the invention can also be characterized by an FT Raman spectrum with characteristic peaks at about 2931, 1379, 512, and 469 cm^{-1.}

Duloxetine HCL crystal Form B in accordance with the invention is an anhydrous form.

In a further embodiment, the invention is directed to polymorphically pure duloxetine HCl Form B. As used herein, the term “polymorphically pure” means that the Form B duloxetine HCl crystalline contains impurities in an amount of less than about 5 percent by weight, based on the total weight of duloxetine HCl. The term “impurities” is defined to include other polymorphic forms of duloxetine HCl, including, but not limited to, Form A.

Preferably, the Form B of duloxetine HCl polymorph has an average particle size of no more than about 500 μm, more preferably, no more than about 300 μm, more preferably, no more than about 200 μm, and, most preferably, no more than about 100 μm. A particularly preferred Form B duloxetine HCl polymorph has an average particle size of no more than about 50 μm.

As used herein, the term “average particle size” refers to the average particle diameter, which may be measured by any method commonly known in the art, including, but not limited to, sieves, sedimentation, electrozone sensing (coulter counter), microscopy, or Low Angle Laser Light Scattering (LALLS).

In another embodiment, the present invention provides a method of preparing duloxetine HCl crystal Form B, comprising providing a solution of duloxetine HCl in water and a solvent selected from the group consisting of C₁₋₄ alcohols, and removing the solvent to obtain duloxetine HCl crystal Form B.

Preferably, the solvent is selected from a group consisting of methanol and ethanol. Most preferably, the solvent is methanol.

Preferably, before removing the solvent, the solution is maintained while stirring. More preferably, the solution is maintained while stirring at about room temperature for about 15 minutes.

Preferably, the solvent is removed by evaporation. More preferably, the solvent is evaporated to dryness at a temperature of from about 35° to about 45° C.

Duloxetine HCl crystal Form B may be recovered by any method known in the art, such as drying the particles, preferably at a temperature of from about 40° C. to about 53° C. under reduced pressure.

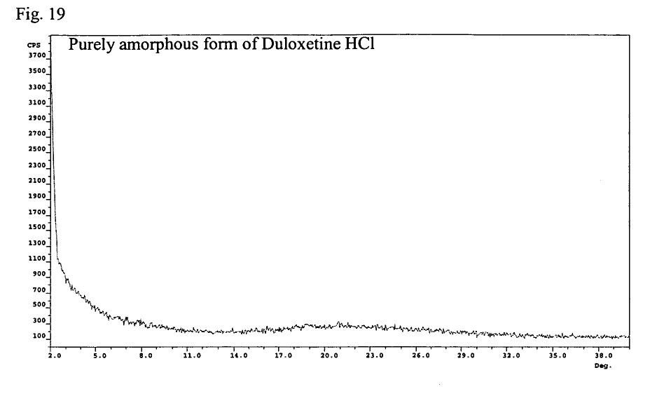

In another embodiment, the present invention provides a process of preparing purely amorphous form of duloxetine HCl. The broad powder X-ray diffraction pattern of the purely amorphous form of duloxetine HCl is illustrated in FIG. 19. This process comprises spray drying a solution of duloxetine HCl in a solvent selected from the group consisting of C₁₋₄ alcohols, where the inlet temperature is ambient, and the outlet temperature is less than the inlet temperature.

Preferably, the solvent is selected from a group consisting of methanol and ethanol. Most preferably, the solvent is methanol.

The spray drying may preferably be conducted with an outlet temperature of below about 20° C., and more preferably about 18° C.

Spray drying broadly refers to processes involving breaking up liquid mixtures into small droplets (atomization), and rapidly removing solvent from the mixture. In a typical spray drying apparatus, there is a strong driving force for evaporation of solvent from the droplets, which may be provided by providing a drying gas. Spray drying processes and equipment are described in Perry's Chemical Engineer's Handbook, pp. 20-54 to 20-57 (6th ed. 1984) and Remington: The Science and Practice of Pharmacy, 19th ed., vol. II, pg. 1627, which are herein incorporated by reference.

By way of non-limiting example only, the typical spray drying apparatus comprises a drying chamber, atomizing means for atomizing a solvent-containing feed into the drying chamber, a source of drying gas that flows into the drying chamber to remove solvent from the atomized-solvent-containing feed, an outlet for the products of drying, and product collection means located downstream of the drying chamber. Examples of such apparatuses include Niro Models PSD-1, PSD-2 and PSD-4 (Niro A/S, Soeborg, Denmark). Typically, the product collection means includes a cyclone connected to the drying apparatus. In the cyclone, the particles produced during spray drying are separated from the drying gas and evaporated solvent, allowing the particles to be collected. A filter may also be used to separate and collect the particles produced by spray drying.

The drying gas used in the process of the present invention may be any suitable gas, although inert gases such as nitrogen, nitrogen-enriched air, and argon are preferred.

The duloxetine HCl product produced by spray drying may be recovered by techniques commonly used in the art, such as using a cyclone or a filter.

In another embodiment, the present invention provides pharmaceutical compositions comprising duloxetine HCl crystal Form B.

Pharmaceutical compositions may be prepared as medicaments to be administered orally, parenterally, rectally, transdermally, bucally, or nasally. Suitable forms for oral administration include tablets, compressed or coated pills, dragees, sachets, hard or gelatin capsules, sub-lingual tablets, syrups, and suspensions. Suitable forms of parenteral administration include an aqueous or non-aqueous solution or emulsion, while for rectal administration, suitable forms for administration include suppositories with hydrophilic or hydrophobic vehicle. For topical administration, the invention provides suitable transdermal delivery systems known in the art, and, for nasal delivery, there are provided suitable aerosol delivery systems known in the art.

In addition to the active ingredient(s), the pharmaceutical compositions of the present invention may contain one or more excipients or adjuvants. Selection of excipients and the amounts to use may be readily determined by the formulation scientist based upon experience and consideration of standard procedures and reference works in the field.

Diluents increase the bulk of a solid pharmaceutical composition, and may make a pharmaceutical dosage form containing the composition easier for the patient and care giver to handle. Diluents for solid compositions include, for example, microcrystalline cellulose (e.g. Avicel®), microfine cellulose, lactose, starch, pregelitinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g. Eudragit®), potassium chloride, powdered cellulose, sodium chloride, sorbitol, and talc.

Solid pharmaceutical compositions that are compacted into a dosage form, such as a tablet, may include excipients whose functions include helping to bind the active ingredient and other excipients together after compression. Binders for solid pharmaceutical compositions include acacia, alginic acid, carbomer (e.g. carbopol), carboxymethylcellulose sodium, dextrin, ethyl cellulose, gelatin, guar gum, hydrogenated vegetable oil, hydroxyethyl cellulose, hydroxypropyl cellulose (e.g. Klucel®), hydroxypropyl methyl cellulose (e.g. Methocel®), liquid glucose, magnesium aluminum silicate, maltodextrin, methylcellulose, polymethacrylates, povidone (e.g. Kollidon®, Plasdone®), pregelatinized starch, sodium alginate, and starch.

The dissolution rate of a compacted solid pharmaceutical composition in the patient's stomach may be increased by the addition of a disintegrant to the composition. Disintegrants include alginic acid, carboxymethylcellulose calcium, carboxymethylcellulose sodium (e.g. Ac-Di-Sol®, Primellose®), colloidal silicon dioxide, croscarmellose sodium, crospovidone (e.g. Kollidon®, Polyplasdone®), guar gum, magnesium aluminum silicate, methyl cellulose, microcrystalline cellulose, polacrilin potassium, powdered cellulose, pregelatinized starch, sodium alginate, sodium starch glycolate (e.g. Explotab®), and starch.

Glidants can be added to improve the flowability of a non-compacted solid composition and to improve the accuracy of dosing. Excipients that may function as glidants include colloidal silicon dioxide, magnesium trisilicate, powdered cellulose, starch, talc, and tribasic calcium phosphate.

When a dosage form such as a tablet is made by the compaction of a powdered composition, the composition is subjected to pressure from a punch and die. Some excipients and active ingredients have a tendency to adhere to the surfaces of the punch and die, which can cause the product to have pitting and other surface irregularities. A lubricant can be added to the composition to reduce adhesion and ease the release of the product from the die. Lubricants include magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc, and zinc stearate.

Flavoring agents and flavor enhancers make the dosage form more palatable to the patient. Common flavoring agents and flavor enhancers for pharmaceutical products that may be included in the composition of the present invention include maltol, vanillin, ethyl vanillin, menthol, citric acid, fumaric acid, ethyl maltol, and tartaric acid.

Solid and liquid compositions may also be died using any pharmaceutically acceptable colorant to improve their appearance and/or facilitate patient identification of the product and unit dosage level.

In liquid pharmaceutical compositions of the present invention, the active ingredient and any other solid excipients are suspended in a liquid carrier, such as water, vegetable oil, alcohol, polyethylene glycol, propylene glycol, or glycerin.

Liquid pharmaceutical compositions may contain emulsifying agents to disperse uniformly throughout the composition an active ingredient or other excipient that is not soluble in the liquid carrier. Emulsifying agents that may be useful in liquid compositions of the present invention include, for example, gelatin, egg yolk, casein, cholesterol, acacia, tragacanth, chondrus, pectin, methyl cellulose, carbomer, cetostearyl alcohol, and cetyl alcohol.

Liquid pharmaceutical compositions of the present invention may also contain a viscosity enhancing agent to improve the mouth-feel of the product and/or coat the lining of the gastrointestinal tract. Such agents include acacia, alginic acid bentonite, carbomer, carboxymethylcellulose calcium or sodium, cetostearyl alcohol, methyl cellulose, ethylcellulose, gelatin guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth, and xanthan gum.

Sweetening agents such as sorbitol, saccharin, sodium saccharin, sucrose, aspartame, fructose, mannitol, and invert sugar may be added to improve the taste.

Preservatives and chelating agents such as alcohol, sodium benzoate, butylated hydroxy toluene, butylated hydroxyanisole, and ethylenediamine tetraacetic acid may be added at levels safe for ingestion to improve storage stability.

According to the present invention, a liquid composition may also contain a buffer such as gluconic acid, lactic acid, citric acid or acetic acid, sodium gluconate, sodium lactate, sodium citrate, or sodium acetate.

Selection of excipients and the amounts used may be readily determined by the formulation scientist based upon experience and consideration of standard procedures and reference works in the field.

The solid compositions of the present invention include powders, granulates, aggregates, and compacted compositions. The dosages include dosages suitable for oral, buccal, rectal, parenteral (including subcutaneous, intramuscular, and intravenous), inhalant, and ophthalmic administration. Although the most suitable administration in any given case will depend on the nature and severity of the condition being treated, the most preferred route of the present invention is oral. The dosages may be conveniently presented in unit dosage form and prepared by any of the methods well known in the pharmaceutical arts.

Dosage forms include solid dosage forms like tablets, powders, capsules, suppositories, sachets, troches, and losenges, as well as liquid syrups, suspensions, and elixirs.

The dosage form of the present invention may be a capsule containing the composition, preferably a powdered or granulated solid composition of the invention, within either a hard or soft shell. The shell may be made from gelatin, and, optionally, contain a plasticizer such as glycerin and sorbitol, and an opacifying agent or colorant.

The active ingredient and excipients may be formulated into compositions and dosage forms according to methods known in the art.

A composition for tableting or capsule filling may be prepared by wet granulation. In wet granulation, some or all of the active ingredients and excipients in powder form are blended, and then further mixed in the presence of a liquid, typically water, that causes the powders to clump into granules. The granulate is screened and/or milled, dried, and then screened and/or milled to the desired particle size. The granulate may then be tableted or other excipients may be added prior to tableting, such as a glidant and/or a lubricant.

A tableting composition may be prepared conventionally by dry blending. For example, the blended composition of the actives and excipients may be compacted into a slug or a sheet, and then comminuted into compacted granules. The compacted granules may subsequently be compressed into a tablet.

As an alternative to dry granulation, a blended composition may be compressed directly into a compacted dosage form using direct compression techniques. Direct compression produces a more uniform tablet without granules. Excipients that are particularly well suited for direct compression tableting include microcrystalline cellulose, spray dried lactose, dicalcium phosphate dihydrate and colloidal silica. The proper use of these and other excipients in direct compression tableting is known to those in the art with experience and skill in particular formulation challenges of direct compression tableting.

A capsule filling of the present invention may comprise any of the aforementioned blends and granulates that were described with reference to tableting, however, they are not subjected to a final tableting step.

While it is apparent that the invention disclosed herein is well calculated to fulfill the objects stated above, it will be appreciated that numerous modifications and embodiments may be devised by those skilled in the art. Therefore, it is intended that the appended claims cover all such modifications and embodiments as falling within the true spirit and scope of the present invention.

### EXAMPLES

While the present invention is described with respect to particular examples and preferred embodiments, it is understood that the present invention is not limited to these examples and embodiments. The present invention, therefore, includes variations from the particular examples and preferred embodiments described herein, as will be apparent to one of skill in the art.

The X-ray diffraction diffractometer used to analyze and identify the crystalline forms of duloxetine HCl was a Scintag X-ray powder diffractometer model X'TRA, Cu-tube solid state detector. The sample holder was a standard round aluminum sample holder with a rough zero background quartz plate, having a cavity diameter of 25 mm and a depth of 0.5 mm. The scanning parameters were:

range: 2 to 40° 2θ;

scan mode: continuous scan;

step size: 0.05 deg.; and

rate: 3 deg/minute.

The FTIR spectrometer used to analyze and identify the crystalline forms of duloxetine HCl was a Perkin-Elmer Spectrum One Spectrometer, incorporating the Diffuse Reflectance Accessory. Samples were finely ground with potassium bromide, and spectra were recorded using a diffuse reflectance technique and a potassium bromide background in the Diffused Reflectance Accessory. The scanning parameters were:

Wavelength range: 4000 to 400 cm⁻¹;

Scans: 16 scans; and

Resolution: 4.0 cm¹.

The FTRaman spectrometer used to analyze and identify the crystalline forms of duloxetine HCl was a Bruker RFS-100/S Raman spectrometer. The scanning parameters were:

Range: 3500 to 50 cm⁻¹;

Aperture Setting: 10.0 mm;

Low Pass Filter 16: 1 kHz;

Source Setting Laser: 9394.0 cm⁻¹, 1600 mW;

Raman Laser Power: 500 mW;

Scanner: Velocity 5.0 at 4 kHz;

Sample Scans:100; and

Resolution: 4.0 cm⁻¹.

Typically, for a determination of weight loss (LOD) by Thermal Gravimetric Analysis (TGA), a sample was heated from about 25° C. to about 200° C. at a heating rate of about 10° C. per minute, while purging with nitrogen gas at a flow rate of 40 ml/minute.

### Example 1

Fifty milliliters of water were added to a solution of 2 g of duloxetine hydrochloride in 25 ml methanol. The solution was stirred at room temperature for fifteen minutes, and the solvent evaporated at 45° C. under vacuum to give the wet solid, which was analyzed by XRD, to be duloxetine HCL Form B. The XRD data is provided in FIG. 2.

### Example 2

Fifty milliliters of water were added to a solution of 2 g of duloxetine hydrochloride in 25 ml of methanol. The solution was stirred at room temperature for fifteen minutes, evaporated to dryness at 45° C. under vacuum, and dried in a vacuum oven at 40° C. for 15 hours. The resulting solid was analyzed by XRD, to be duloxetine HCL Form B. The XRD data is provided in FIG. 2.

### Example 3

Ten grams of duloxetine hydrochloride was dissolved in 250 ml of methanol, and the resulting solution was sprayed at a rate of 72 ml/hour into a chamber with ambient nitrogen at a co-current flow of 38 m³/hour and a temperature of 42° C. The atomizing flow of nitrogen at 660 l/hour produced droplets, leading to a high evaporation rate. The temperature of the outlet solids was fixed at 32° C. The resulting powder was analyzed using XRD, and found to be the purely amorphous form.

### Example 4

Ten grams of duloxetine hydrochloride was dissolved in 250 ml of methanol, and the resulting solution was sprayed at 145 ml/hour into a chamber with ambient nitrogen at a co-current flow of 38 m³/hour and a temperature of 27° C. The atomizing flow of nitrogen at 440 l/hour produced droplets, leading to a high evaporation rate. The temperature of the outlet solids was fixed at 18° C. The resulting powder was analyzed by XRD, and found to be the purely amorphous form.

What is claimed:

1. A crystalline form of duloxetine hydrochloride, characterized by at least one of: an X-ray powder diffraction pattern having peaks at about 11.1, 12.1, 14.9, 16.3, 21.6, 24.2, 27.1, and 30.0 degrees two-theta ±0.2 degrees two-theta; an IR spectrum having peaks at about 1093, 1024, 797, and 778 cm⁻¹; and a Raman spectrum having peaks at about 2931, 1379, 512, and 469 cm⁻¹.

2. The crystalline form of claim 1, characterized by X-ray powder diffraction pattern having peaks at about 11.1, 12.1, 14.9, 21.6 and 24.2 degrees two-theta ±0.2 degrees two-theta.

3. The crystalline form of claim 2, further characterized by X-ray powder diffraction pattern having peaks at about 21.6 and 30.0 degrees two-theta ±0.2 degrees two-theta.

4. The crystalline form of claim 3, characterized by an XRD pattern substantially identified by FIG. 2.

5. The crystalline form of claim 1, characterized by IR spectrum having peaks at about 1093, 1024, 797, and 778 cm⁻¹.

6. The crystalline form of claim 5, characterized by an IR spectrum substantially identified by FIGS. 7-10.

7. The crystalline form of claim 1, characterized by a Raman spectrum having peaks at about 2931, 1379, 512, and 469 cm⁻¹.

8. The crystalline form of claim 7, characterized by a Raman spectrum substantially identified by FIGS. 15-18.

9. The crystalline form of claim 1, comprising less than about 5 percent by weight of other polymorphic forms of duloxetine hydrochloride.

10. A method of preparing the crystalline form of claim 1 comprising:
a. providing a solution of duloxetine HCl in water and a solvent selected from the group consisting of C₁₋₄ alcohols ; and
b. removing the solvent, to obtain the crystalline form of claim 1.

11. The method of claim 10, wherein the solvent is selected from a group consisting of methanol and ethanol.

12. The method of claim 11, wherein the solvent is methanol.

13. The method of claim 10, wherein the solvent is removed by evaporation.

14. The method of claim 13, wherein the solvent is evaporated to dryness at a temperature of from about 35° to about 45° C.

15. A process of preparing purely amorphous form of duloxetine HCl comprising spray drying a solution of duloxetine HCl in a solvent selected from the group consisting of C₁₋₄ alcohols, wherein the spray has an ambient inlet temperature and an outlet temperature less than the inlet temperature.

16. The process of claim 15, wherein the solvent is selected from the group consisting of methanol and ethanol.

17. The process of claim 16, wherein the solvent is methanol.

18. The process of claim 15, wherein the outlet temperature is below about 20° C.

19. The process of claim 18 wherein the outlet temperature is 18° C.

20. A pharmaceutical composition, comprising the crystalline form of claim 1.
